(19) United States
(12) Patent Application Publication
(10) Pub. No.: US 2025/0068239 A1
(43) Pub. Date: Feb. 27, 2025

(54) GAIN ADJUSTMENT USING CONTACT DETECTION

(71) Applicant: Apple Inc., Cupertino, CA (US)

(72) Inventors: Tao Shui, Cupertino, CA (US); Adriaan J. Taal, Mountain View, CA (US); Na Huang, Cupertino, CA (US)

(21) Appl. No.: 18/776,052

(22) Filed: Jul. 17, 2024

Related U.S. Application Data
(60) Provisional application No. 63/534,459, filed on Aug. 24, 2023.

(51) Int. Cl. G06F 3/01; G06F 1/16; G06F 3/0488

(52) U.S. Cl. CPC G06F 3/015; G06F 1/163; G06F 3/014; G06F 3/0488

(57) ABSTRACT

Embodiments are directed to methods, devices and systems for determining and applying a gain adjustment to sensed electrical signals. The methods can include operating a device to determine that a user is wearing the wearable electronic device and applying a test signal to a sensing electrode of the wearable device. An input impedance for the sensing electrode can be determined using the test signal. The input impedance and a baseline impedance can be used to determine a gain adjustment. An electrical signal of the user can be measured using the sensing electrode and the gain adjustment can be applied to the electrical signal. A physiological parameter of the user can be determined using the measured electrical signal having the gain adjustment applied.

### CROSS-REFERENCE TO RELATED APPLICATION

This application is a nonprovisional and claims the benefit under 35 U.S.C. 119(e) of U.S. Provisional Patent Application No. 63/534,459, filed Aug. 24, 2023, the contents of which are incorporated herein by reference as if fully disclosed herein.

### FIELD

The described embodiments relate generally to devices and methods for processing physiological signals, and more particularly, the present embodiments relate to adjusting physiological signals based on contact metrics of one or more electrodes with a user.

### BACKGROUND

Electrodes may be used as part of electronic devices to measure electric potentials of a user. For example, electrocardiogram (ECG) measurements can use electrodes that contact a user's body to measure cardiac electrical activity of a user over one or more cardiac cycles. Similarly, electromyography (EMG) and electroencephalography (EEG) can use electrodes to measure muscle and brain activity, respectively. Electrodes may contact a user at different portions of a user's body and a pair of electrodes can each measure an electrical signal at different locations on the user and the measured signals from the different electrodes can be used to determine an electrical potential difference. In an ECG measurement, the electrical potential difference over one or more cardiac cycles can be analyzed to obtain information about the electrical functioning of the user's heart.

Traditional electrodes, which may be referred to as “wet electrodes,” include an electrically conductive gel to facilitate electrical conduction from the skin. Electrodes may be incorporated into wearable devices such as a smartwatch, which may be used to continuously or periodically monitor cardiac electrical activity of a user. It may be desirable to utilize “dry electrodes,” which do not require an electrically conductive gel, in wearable device such as a smartwatch. Dry electrodes may result in greater signal attenuation of electrical signals from the user's skin.

### SUMMARY

Embodiments are directed to methods for electrode sensing at a wearable electronic device. The methods can include, in response to determining that a user is wearing the wearable electronic device applying, using the contact detection circuit, a test signal to the sensing electrode and determining, using the contact detection circuit, an input impedance for the sensing electrode using the test signal. The methods can include determining a gain adjustment using the input impedance and a baseline impedance and measuring, using a physiological sensing circuit, an electrical signal of the user using the sensing electrode. The methods can include applying, using the physiological sensing circuit, the gain adjustment to the measured electrical signal and determining a physiological parameter of the user using the measured electrical signal having the gain adjustment applied.

Embodiments are also directed to methods for electrode gain calibration of sensing electrode signals at a wearable electronic device. The methods can include isolating a contact detection circuit from a physiological sensing circuit. While the contact detection circuit is isolated from the physiological sensing circuit, a first test signal can be applied to a sensing electrode of the wearable electronic device. The methods can include determining a first input impedance for the sensing electrode using the first test signal. The contact detection circuit can be connected to the physiological sensing circuit. The methods can include applying, while the contact detection circuit is connected to the physiological sensing circuit, a second test signal to the sensing electrode. A second input impedance can be determined for the sensing electrode using the second test signal. The methods can include determining a gain adjustment using the first input impedance and the second input impedance, measuring an electrical signal of a user using the sensing electrode, and determining a physiological parameter of the user by applying the gain adjustment to the measured electrical signal.

Embodiments further include a system for electrode sensing at a wearable electronic device. The system can include a housing configured to be coupled to a user, a sensing electrode coupled to the housing and configured to contact the user and a processor. The processor can be configured to, in response to determining that the user is wearing the wearable electronic device, cause a test signal to be applied to the sensing electrode and determine an input impedance for the sensing electrode using the test signal. The processor can determine a gain adjustment using the input impedance and a baseline impedance, measure an electrical signal of the user using the sensing electrode, and determine a physiological parameter of the user by applying the gain adjustment to the measured electrical signal.

### BRIEF DESCRIPTION OF THE DRAWINGS

The disclosure will be readily understood by the following detailed description in conjunction with the accompanying drawings, wherein like reference numerals designate like structural elements, and in which:

FIG. 1A shows a front view of an example electronic device that can be used to perform physiological measurements, as described herein;

FIG. 1B shows a back view of an example electronic device that can be used to perform physiological measurements, as described herein;

FIG. 2 shows an example block diagram for an electronic device that can be used to perform physiological measurements, as described herein;

FIG. 3 shows an example gain adjustment circuit that can be used to perform physiological measurements, as described herein;

FIG. 4 shows an example contact detection circuit that can be used to perform physiological measurements, as described herein;

FIG. 5 shows an example process for performing physiological measurements using a gain adjustment circuit;

FIG. 6 shows an example gain calibration circuit that can be used to perform physiological measurements, as described herein; and

FIG. 7. shows an example process for performing physiological measurements using a gain calibration circuit.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications and equivalents as can be included within the spirit and scope of the described embodiments as defined by the appended claims.

Embodiments disclosed herein are directed to devices, systems and methods for improving the accuracy of physiological measurements, and in particular, physiological measurements made using electrodes that contact the skin of a user. The devices and systems described herein can include electrodes which can sense electrical signals from a skin surface of a user and/or apply electrical signals to a skin surface of a user. The devices and systems may include one or more circuits which help improve the accuracy of electrical measurements by determining and applying a gain adjustment to electrical signals sensed by one or more electrodes. For example, the devices and systems may perform electrocardiogram (ECG) measurements, which can include using one or more electrodes to sensing cardiac electrical signals of a user and applying a gain adjustment to sensed cardiac electrical signals.

In some cases, the user-electrode interface may have high electrical impedance, which can attenuate signals sensed by an electrode. For example, when dry electrodes are used to measure electrical signals of the user, the contact between the dry electrode and the user's skin can have high input impedance. As used herein, the term “dry electrode” refers to electrodes that do not use a gel or other liquid material to form a conductive path between the skin and an electrode. In some cases, the input impedance may cause signal attenuation that results in physiological measurements (e.g., ECG measurements) that do not meet at least one particular accuracy and/or reliability criterion. For example, a physiological parameter such as an ECG measurement that is determined from a highly attenuated signal may not meet a defined accuracy and/or reliability criterion. That is, the device may determine that the quality of the signal is too low to accurately determine an ECG measurement for the user.

Embodiments described herein include systems, devices and methods for determining and applying a gain adjustment to measured physiological signals of a user. In some cases, the system can determine a gain adjustment using system calibration data and user-specific calibration data. System calibration data may represent general properties of the system itself, which may include baseline impedance data for one or more electrodes. User-specific calibration data can relate to the quality of electrode contact with a user and may include impedance data determined from a test signal applied to an electrode while the electrodes are in contact with the user. The system calibration data for a sensing electrode may be determined prior to a user wearing a device or during an initial use of the device by a user, for example as part of a manufacturing process, and is stored by the system. The system calibration data may be obtained, for example, by applying a first test signal to the sensing electrode and using a contact detection circuit to determine a baseline impedance for the sensing electrode. The first test signal may be applied using any suitable method and does not necessarily need to mimic biological conditions of a user. For example, the first test signal may be applied using a test probe and the determined baseline impedance may be lower than an expected impedance due to contact with a user. The user-specific calibration data may be obtained by determining that a user is wearing the electronic device and using the contact detection circuit to apply a second test signal to the user and determining a second input impedance based on the contact of the sensing electrode with the user. In some cases, the user-specific calibration data can be determined using contact detection processes and hardware discussed in U.S. Patent Application Publication Number 2020/0077955, the contents of which are incorporated herein by reference.

The determined gain adjustment may be used to increase the power and/or amplitude of signals measured by the sensing electrode. For example, the sensing electrode may be operated to measure cardiac electrical signals of a user and determine one or more ECG parameters of a user. The device may apply the determined gain adjustment to the measured cardiac signals to increase a power and/or amplitude of the measured cardiac signals. The device may determine the one or more ECG parameters by applying the gain adjustment to the measured cardiac signals.

In some cases, the device may determine whether to apply a gain adjustment to a measured physiological signal. For example, a wearable device may determine whether the user-specific calibration data and/or the determined gain satisfies at least one criterion. The criterion may be an impedance threshold that defines an impedance range where a gain adjustment is applied. In response to satisfying the at least one criterion (e.g., an impedance based on contact of the sensing electrode with the user is within the impedance range), the wearable device applies the gain adjustment to the measured physiological signal. In response to not satisfying the at least one criterion (e.g., an impedance based on contact of the sensing electrode with the user is not within the impedance range), the wearable device does not apply the gain adjustment to the measured physiological signal.

In some cases, the systems and/or devices may include multiple sensing electrodes and determine a gain adjustment for each of the electrodes. For example, the system may determine and apply a first gain adjustment to electrical signals measured by a first sensing electrode and determine and apply a second gain adjustment to electrical signals measured by a second sensing electrode.

Embodiments described herein also include systems, devices and methods for determining and applying gain adjustment to account for impedance changes and/or other changes in one or more circuits that cause attenuation of measured physiological signals of a user. The systems devices may include a contact detection circuit that is used to characterize a contact of the sensing electrode with a user (e.g., determine an input impedance based on the contact of the sensing electrode with the user), and a physiological sensing circuit that is used to measure electrical signals of a user. The systems and/or devices may be operated to isolate the contact detection circuit from the physiological sensing circuit. A first test signal can be applied to a sensing electrode while the physiological detection circuit is isolated from the contact detection circuit. A first impedance for the sensing electrode can be determined, which may correspond to an impedance of the contact detection circuit. The contact detection circuit can be connected to the physiological sensing circuit and a second test signal can be applied to the sensing electrode. A second impedance can be determined for the sensing electrode and may correspond to an impedance of the contact detection circuit and the physiological sensing circuit. A gain adjustment can be determined from the first and second impedances and the determined gain adjustment can be applied to measured electrical signals of the user to compensate for attenuation or other signal degradation due to the contact detection circuit and/or physiological sensing circuit.

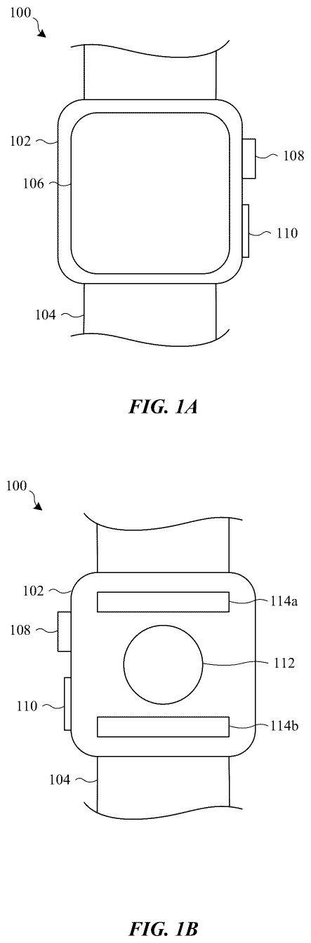

These and other embodiments are discussed below with reference to FIGS. 1-7. However, those skilled in the art will readily appreciate that the detailed description given herein with respect to these Figures is for explanatory purposes only and should not be construed as limiting.

FIG. 1A shows a front view of an example electronic device 100 and FIG. 1B shows a back view of the example electronic device 100 which can be used to perform physiological measurements, as described herein. The electronic device 100 is depicted as a watch, though this is merely one example embodiment of an electronic device, and the concepts discussed herein may apply equally or by analogy to other electronic devices, including mobile phones (e.g., smartphones), tablet computers, notebook computers, head-mounted displays, headphones, earbuds, digital media players (e.g., mp3 players), wearable bands, rings, or the like.

The device 100 includes a housing 102 and a band 104 coupled to the housing. The housing 102 may at least partially define an internal volume in which components of the device 100 may be positioned. The housing 102 may also define one or more exterior surfaces of the device, such as all or a portion of one or more side surfaces, a rear surface, a front surface, and the like. The housing 102 may be formed of any suitable material, such as metal (e.g., aluminum, steel, titanium, or the like), ceramic, polymer, glass, or the like.

The band 104 may attach the device 100 to a user, such as to the user's arm or wrist. The device 100 may include battery charging components within the device 100, which may receive power, charge a battery of the device 100, and/or provide direct power to operate the device 100 regardless of the battery's state of charge (e.g., bypassing the battery of the device 100). The device 100 may include a magnet, such as a permanent magnet, that magnetically couples to a magnet (e.g., a permanent magnet, electromagnet) or magnetic material (e.g., a ferromagnetic material such as iron, steel, or the like) in a charging dock (e.g., to facilitate wireless charging of the device 100).

The electronic device 100 can include a display 106. The display 106 can be positioned at least partially within the housing 102. The display 106 may define an output region in which graphical outputs are displayed. Graphical outputs may include graphical user interfaces, user interface elements (e.g., buttons, sliders, etc.), text, lists, photographs, videos, or the like. In some cases, the display 106 may output a graphical user interface with one or more graphical objects that display information collected from or derived from one or more sensors. For example, the display 106 may output one or more physiological paraments, such as ECG parameters that were measured for a user.

The display 106 may include or be associated with touch sensors and/or force sensors that extend along the output region of the display and which may use any suitable sensing elements and/or sensing techniques. Using touch sensors, the electronic device 100 may detect touch inputs applied to the cover, including detecting locations of touch inputs, motions of touch inputs (e.g., the speed, direction, or other parameters a gesture applied to the cover can generate), or the like. Using force sensors, the electronic device 100 may detect amounts or magnitudes of force associated with touch events applied to the cover. The touch and/or force sensors may detect various types of user inputs to control or modify the operation of the electronic device 100, including taps, swipes, multiple finger inputs, single- or multiple-finger touch gestures, presses, and the like.

The electronic device 100 may also include one or more user inputs such as a first input device 108 having a cap, crown, protruding portion, or component(s) or feature(s) positioned along a side surface of the housing 102. At least a portion of the first input device 108 (such as a crown body) may protrude from, or otherwise be located outside, the housing 102, and may define a generally circular shape or circular exterior surface. The exterior surface of the first input device 108 may be textured, knurled, grooved, or otherwise have features that may improve the tactile feel of the first input device 108 and/or facilitate rotation sensing.

The first input device 108 may facilitate a variety of potential interactions. For example, the first input device 108 may be rotated by a user (e.g., the crown may receive rotational inputs). Rotational inputs of the first input device 108 may zoom, scroll, rotate, or otherwise manipulate a user interface or other object displayed on the display 106 among other possible functions. The first input device 108 may also be translated or pressed (e.g., axially) by the user. Translational or axial inputs may select highlighted objects or icons, cause a user interface to return to a previous menu or display, or activate or deactivate functions among other possible functions.

In some cases, the electronic device 100 may sense touch inputs or gestures applied to the first input device 108, such as a finger sliding along the body of the first input device 108 (which may occur when first input device 108 is configured to not rotate) or a finger touching the body of the first input device 108. In such cases, sliding gestures may cause operations similar to the rotational inputs, and touches on a cap or crown may cause operations similar to the translational inputs. As used herein, rotational inputs include both rotational movements of the first input device 108, as well as sliding inputs that are produced when a user slides a finger or object along the surface of a crown in a manner that resembles a rotation (e.g., where the crown is fixed and/or does not freely rotate).

The electronic device 100 may also include other input devices, switches, buttons, or the like. For example, the electronic device 100 includes a second input device 110, which may be a button. The second input device 110 may be a movable button or a touch-sensitive region of the housing 102. The button may control various aspects of the electronic device 100. For example, the button may be used to select icons, items, or other objects displayed on the display 106, to activate or deactivate functions (e.g., to silence an alarm or alert), or the like.

FIG. 1B shows a rear side of the device 100. The electronic device 100 may include one or more windows 112 (one of which is shown) that allow light to pass through a portion of the housing 102. The one or more windows 112 may be coupled to the housing 102. The one or more windows 112 may include light transmissive materials and be associated with internal sensor components, which may be used to determine biometric information of a user, such as heart rate, blood oxygen concentrations, and the like, as well as information such as a distance from the device to an object. The particular arrangement of the one or more windows 112 in the housing 102 shown in FIG. 1B is one example arrangement, and other window arrangements (including different numbers, sizes, shapes, and/or positions of the windows) are also contemplated. As described herein, the window arrangement may be defined by or otherwise correspond to the arrangement of components in the integrated sensor package.

The housing 102 may also include one or more electrodes 114a, 114b (collectively electrodes 114). The electrodes 114 may facilitate input to biometric sensing circuitry or other sensing circuitry within the device 100. The electrodes 114 may be a conductive surface that is conductively coupled, via one or more components of the device 100, to the biometric sensing circuitry. In some cases, one or more input devices (e.g., the first input device 108 and/or the second input device 110) may be operated as an electrode that is used to apply electrical signals to a user and/or sense electrical signal from a user. The input devices may be conductively coupled to sensing circuitry within the housing 102.

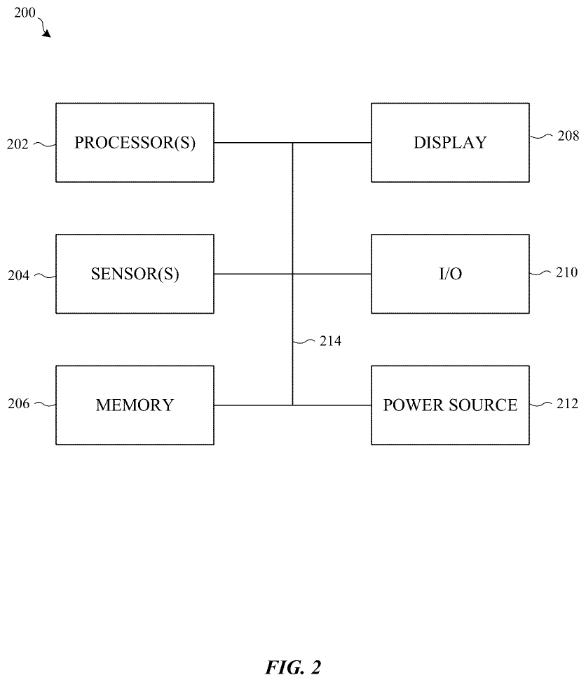

FIG. 2 shows an example block diagram 200 for an electronic device that can be used to perform physiological measurements, as described herein. The electronic device 200 can include a processor 202, one or more sensors 204, memory 206, a display 208, an input/output (I/O) mechanism 210, and a power source 212.

The processor 202 can control some or all of the operations of the electronic device 200. The processor 202 can communicate, either directly or indirectly, with some or all of the components of the electronic device 200. For example, a system bus or other communication mechanism 216 can provide communication between, the processor 202, the one or more sensors 204, the memory 206, the display 208, the input/output (I/O) mechanism 210, and the power source 212.

The processor 202 can be implemented as any electronic device capable of processing, receiving, or transmitting data or instructions. For example, the processor 202 can be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitable computing element or elements.

It should be noted that the components of the electronic device 200 can be controlled by multiple processors. For example, select components of the electronic device 200 (e.g., a sensor 204) may be controlled by a first processor and other components of the electronic device 200 (e.g., the I/O 210) may be controlled by a second processor, where the first and second processors may or may not be in communication with each other.

The electronic device 200 may also include one or more sensors 204 positioned almost anywhere on the electronic device 200. The sensor(s) 204 can be configured to sense one or more type of parameters, such as but not limited to, electrical signals, pressure, sound, light, touch, heat, movement, relative motion, biometric data (e.g., physiological parameters), and so on. For example, the sensor(s) 204 may include, one or more electrodes (and corresponding circuitry), a pressure sensor, an auditory sensor, a heat sensor, a position sensor, a light or optical sensor, an accelerometer, a pressure transducer, a gyroscope, a magnetometer, a health monitoring sensor, and so on. Additionally, the one or more sensors 204 can utilize any suitable sensing technology, including, but not limited to, capacitive, ultrasonic, resistive, optical, ultrasound, piezoelectric, and thermal sensing technology.

The memory 206 can store electronic data that can be used by the electronic device. For example, the memory 206 can store electrical data or content such as, for example, measured electrical signals, audio and video files, documents and applications, device settings and user preferences, timing signals, control signals, and data structures or databases. The memory 206 can be configured as any type of memory. By way of example only, the memory 206 can be implemented as random access memory, read-only memory, Flash memory, removable memory, other types of memory storage elements, or combinations of such devices.

The electronic device 200 may also include a display 208. The display 208 may include a liquid-crystal display (LCD), organic light-emitting diode (OLED) display, light-emitting diode (LED) display, or the like. If the display 208 is an LCD, the display 208 may also include a backlight component that can be controlled to provide variable levels of display brightness. If the display 208 is an OLED or LED type display, the brightness of the display 208 may be controlled by modifying the electrical signals that are provided to display elements. The display 208 may correspond to any of the displays shown or described herein.

The I/O mechanism 210 can transmit and/or receive data from a user or another electronic device. An I/O mechanism 210 can include a display, a touch sensing input surface, one or more buttons (e.g., a graphical user interface “home” button), one or more cameras, one or more microphones or speakers, one or more ports, such as a microphone port, and/or a keyboard. Additionally or alternatively, an I/O device or port can transmit electronic signals via a communications network, such as a wireless and/or wired network connection. Examples of wireless and wired network connections include, but are not limited to, cellular, Wi-Fi, Bluetooth, IR, and Ethernet connections.

The power source 212 can be implemented with any device capable of providing energy to the electronic device 200. For example, the power source 212 may be one or more batteries or rechargeable batteries. Additionally or alternatively, the power source 212 can be a power connector or power cord that connects the electronic device 200 to another power source, such as a wall outlet.

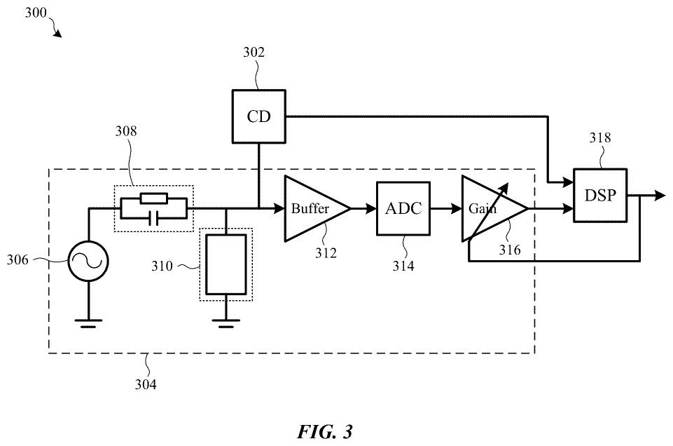

FIG. 3 shows an example gain adjustment circuit 300 that can be used to perform physiological measurements, as described herein. The example gain adjustment circuit 300 can include a contact detection (CD) circuit 302 and a physiological sensing circuit 304. In some cases, the example circuit 300 may be implement on a single application specific integrated circuit (ASIC). In other cases, the example gain adjustment circuit 300 may be implemented across multiple ASICs and various components can be integrated on different ones of the multiple ASICs.

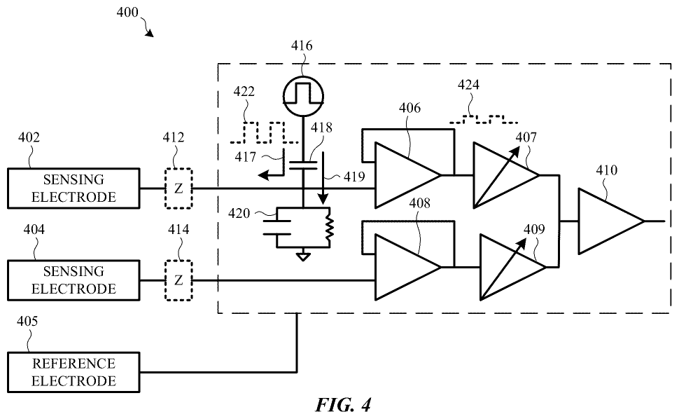

In some cases, the CD circuit 302 can be operated to determine a baseline input impedance, which may be performed at the factory (or as part of a device initiation procedure) and a second, user input impedance, which is performed when a user is contacting an electrode 308. In other cases, baseline input impedance may be determined using other techniques that don't require the use of the contact detection circuit and the baseline input impedance value may be stored at the electronic device. An electrical signal from CD circuit 302 can be applied to the input of buffer 312, such that CD circuit 302 can determine an input impedance for the electrical signal. The determined input impedance can include contact impedance due to impedance at the electrode 308 that results from contact of the electrode with a skin surface of a user and/or contact with a test electrode. The determined input impedance can also include a circuit impedance 310 due to impedance of the detection circuitry. An example circuit diagram for the contact detection circuit is shown in FIG. 4.

The physiological sensing circuit 304 can include a buffer 312 that receives the detected electrical signal 306 from the electrode. The buffer 312 may include a voltage buffer amplifier that is used to transfer sensed voltage from the sensing electrode 308 and downstream circuitry until it is further processed by the circuit 300. The physiological sensing circuit 304 can also include an analog to digital converter (ADC) 314, which can receive the electrical signal(s) from the buffer 312 and convert the analog signals into a digital signal. The digitized signals can be transferred to a gain amplifier 316, which can increase amplitude of the electrical signals. The gain amplifier 316 can be a digital gain amplifier that increases the amplitude of a digital signal received from the analog to digital converter.

The circuit 300 can include a digital signal processor 318 that can receive impedances determined using the contact detection circuit 302 and/or the detected and gain adjusted electrical signals from the physiological sensing circuit 304. The digital signal processor 318 can perform one or more functions on the received signals, such as associating the signals with one or more measurements parameters and transfer the signals to a host processor the electronic device (e.g., electronic device 100, 200), which may generate one or more outputs based on the received signals as described herein.

FIG. 4 shows an example contact detection circuit 400 (e.g., contact detection circuit 302) that can be used to determine an input impedance for an electrode, as described herein. The contact detection circuit can be an example of the systems hardware discussed in U.S. Patent Application Publication Number 2020/0077955, the contents of which are incorporated herein by reference.

FIG. 4 illustrates circuit components that may be implemented on one or more ASICs and/or using other hardware. FIG. 4 focuses on electrode(s) and measurement circuitry that can be used to determine input impedance, however the contact detection circuit and/or electronic device may include other components including processing circuitry and/or additional electrode(s) (e.g., a reference electrode).

The circuit 400 can include a first sensing electrode 402 (e.g., corresponding to the first electrode 114a), a second sensing electrode 404 and a reference electrode 405 (e.g., corresponding to the second electrode 114b). The circuit 400 can include buffers 406 and 408, gain amplifiers 407 and 409 and amplifier 410. The buffers 406 and 408 can provide impedance matching for the electrodes 402 and 404. The buffers 406 and 408 can be configured to accommodate input impedances 412 and 414 that result from the electrodes 402 and 404 contact with a user and/or from the circuit 400. In some cases, the buffers 406 and 408 are configured to reduce noise and/or interference for input to the amplifier 410. In some cases, the amplifier 410 can be coupled to an analog to digital converter and outputs from the amplifier can be fed into the analog to digital converter. Additionally or alternatively, the gain amplifiers 407 and 409 can be operated to provide gain adjustments to electrical signals before the signals are transmitted to amplifier 410, which can be configured as a difference amplifier.

The circuit 400 can also include test signal generator 416, capacitor 418 and an impedance network 420. The signal generator 416 can generate one or more test signals 422 that are used to determine an impedance of the sensing electrode 402, which may result from contact of the sensing electrode 402 with a user. The capacitor 418 and impedance network 420 can form a voltage divider through path 419 to ground and a test signal 422 generated by test signal generator 416 can be divided by the voltage divider. Buffer 406 can measure a node between the capacitor 418 and impedance network 420. A measured test signal 424 can be used to determine an input impedance for the sensing electrode 402.

The amplitude (e.g., voltage level) of the measured test signal 424 can depend on the load experienced by the test signal 422. For example, when a user touches sensing electrode 402, the resulting test signal 422 can be attenuated. Contact between a user (e.g., a finger) and sensing electrode 402 can form a path 417 for test signal 422. In some examples, path 417 can be formed through a physiological signal source (e.g., the body of the user) to system ground via a ground electrode (e.g., a ground electrode contacting the user). In some examples, a user can be contacting sensing electrode 402 with a first portion of their body (e.g., a finger) and the housing of the device with a second portion of their body (e.g., their wrist). In such cases, path 417 for test signal 422 can be formed through physiological signal source (e.g., the body of the user) to system ground through the finger touching the housing of the device (e.g., the housing of the device can be grounded to system ground). Thus, path 417 can form an impedance in parallel to path 419 (through impedance network 412) and change the loading experienced by test signal 422. In such examples, the resulting measured test signal 424 at buffer 406 can be attenuated. In contrast, when a user is not touching sensing electrode 402, the resulting measured test signal 424 may not be attenuated (or may be attenuated less).

Test signal 422 (e.g., stimulation signal) generated by test signal generator 416 can be a square wave, a sine wave, a trapezoidal wave, a saw-tooth wave or any other suitable periodically oscillating, non-oscillating or non-periodic (e.g., pseudo-noise signal) waveform. The test signal, regardless of waveform, can be known or predetermined to the system to enable detection of the resulting measured test signal, in some examples as described herein. Test signal 422 can be capacitively or resistively coupled via capacitor 418 to sensing electrode 402. In some examples, test signal generator 416 can be controlled by a processor (e.g., a digital signal processor (DSP), host processor, or other processor). In some examples, the processor can change the frequency and/or amplitude of test signal 422 and/or enable and disable test signal generator 416. In some examples, the test signal generator 416 can be a clock output of a processor.

In some examples, the response of test signal 422 to the load can depend on the frequency of test signal 422 and the respective impedance of the signal paths. In some examples, the frequency of test signal 422 can be varied to determine the load of the signal paths at the respective frequency (e.g., the quality of the skin-to-electrode connection as a function of the test signal frequency can be determined). In some examples, an initialization process can be used to select a frequency for differentiating between when sensing electrode 402 is contacted and when it is not contacted (e.g., a frequency for test signal 422 that results in an observable change in resulting test signal amplitude). In some examples, test signal 422 can include a plurality of frequencies concurrently (e.g., test signal 422 can include multiple frequency components). In such an example, the reactance of the system to different frequencies can be determined at one time.

In some examples, when a user contacts sensing electrode 402, a physiological signal from the user can enter the circuit 300. In some examples, beginning physiological signal measurements can include acquiring the physiological signal (e.g., by data buffer 316), storing the physiological signal (e.g., in memory) and/or displaying the physiological signal on a display of the device. In some examples, when the system determines that the sensing electrode is not contacted, the system can forego measuring the physiological signal (e.g., powering down the circuit, discarding the physiological signal measurements, or otherwise not process incoming signals). In some examples, when the system determines that the sensing electrode is not contacted, the system can still measure the physiological signal, but with a low confidence value indicative that the physiological signal is low-quality (e.g., may not be reliable for one or more intended uses). In some examples, the low confidence can be represented in a binary manner (e.g., a low-confidence/low-quality flag can be set). In some examples, the confidence can be represented in another manner (e.g., a probability) representative of the quality. In some examples, when the confidence is below a threshold or when the low-confidence/low-quality flag is set, a notification can be presented to the user to indicate that the measured physiological signal measurement may be unreliable or low quality (e.g., display the physiological signal with a visual indicator, display a notification on the display of the device and/or any other visual feedback, and/or an audio feedback and/or a haptic feedback and/or any other suitable feedback mechanism).

Although FIG. 3 illustrates the integration of the contact detection circuit 302 with the physiological sensing circuit 304, it is understood that contact detection circuit can be implemented in a different manner. For example, the test signal circuitry can include an amplifier or other front-end circuitry (e.g., separate from amplifier 410, etc.) to perform the functions of measuring the test signal and performing contact detection. In some examples, separate signal paths can be used for contact detection and physiological sensing (e.g., not integrating the test signal circuitry with the physiological signal measurement circuitry). In some examples, implementing contact detection and physiological sensing separately can allow for optimization of the circuitry for contact detection for the frequencies, signal range and/or signal precision of the test signal for contact detection and optimization of the circuitry for the frequencies, signal range and/or signal precision for physiological sensing. In some examples a switching circuit can be provided to couple the test signal circuitry (e.g., test signal generator and measurement amplifier) to the sensing electrode during contact detection and to decouple the sensing electrode from the test signal circuitry during the physiological signal measurement. In some examples, the test signal circuitry can be integrated with saturation detection circuitry. Additionally, although illustrated as a discrete source in FIG. 4, test signal 422 can be generated by a processor (e.g., DSP, host processor). In some examples, the same processor can also be coupled to receive the measured test signals from the output of buffer 406 and/or 408 or another buffer or amplifier circuit.

Additionally or alternatively, although FIG. 3 and FIG. 4 illustrate the integration of the test signal circuitry into the signal path of a sensing electrode, similar test signal circuitry can be integrated onto the signal path of other sensing electrodes and/or a reference electrode (e.g., reference electrode 405). For example, in the operation described with reference to FIG. 4, the reference electrode 405 may be coupled to a ground circuit. However, in cases where contact detection is performed at the reference electrode 405, a different electrode may be coupled to the ground circuit and the contact detection process can be applied to the reference electrode 405 using the same or similar process for the sensing electrode(s) 402, 404, describe herein. Determining a contact metric for the reference electrode 405 can occur at different times than determining a contact metric for a sensing electrode.

Additionally, although FIG. 4 illustrates sensing electrodes 402, 404 and a reference electrode 405, in some examples, the system can have a plurality of sensing electrodes and/or a plurality of reference electrodes, and similar test signal circuitry can be integrated with some or all of these electrodes.

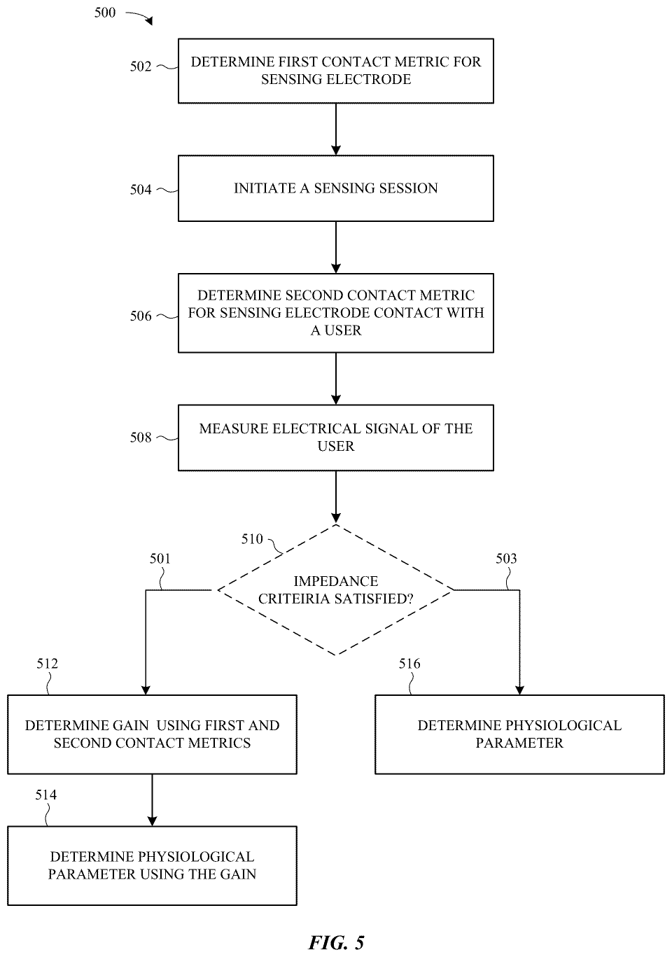

FIG. 5 shows an example process 500 for performing physiological measurements to apply a gain adjustment to a physiological signal. The process can be performed using the circuitry described herein (e.g., circuitry 300 and 400 described with reference to FIG. 3 and FIG. 4).

At operation 502, the process 500 can include determining a first contact metric for a sensing electrode. The first contact metric may be baseline metric that is determined prior to a device being used to perform physiological measurements. In some cases, the first contact metric can be determined from a calibration procedure performed at a factory and/or prior to a user wearing the device.

Determining a first contact metric can include applying one or more test signals to the sensing electrode and measuring an effect of the signal, for example using the circuitry 300 and 400 described herein. In some cases, the test signal can be applied using a calibration probe that contacts the sensing electrode (or other electrode(s)) on the device. For example, the calibration probe may be brought into contact with the sensing electrode and applies the test signal(s) to the sensing electrode. The device, using the contact detection circuit, may determine an impedance and/or other metric (e.g., voltage signal amplitude) for the test signal. In other cases, the calibration probe may be configured to complete a circuit between the sensing electrode, a reference electrode and/or ground electrode and the contact detection circuit can apply the test signal to the circuit. The resulting test signal can be measured and used to determine an impedance metric for the calibration probe.

At operation 504, the process 500 can include initiating a sensing session for a user. Initiating a sensing session can include the device making a determination that the device is being worn by a user and/or one or more electrodes are contacting a user. In some cases, the device may use one or more sensors to make a determination that the user is wearing the device and/or contacting the electrode(s). For example, the device may operate the contact detection circuit to apply a test signal to an electrode and measure the test signal as described herein. The measured test signal can be used to determine if the user is wearing the electronic device and/if there is sufficient contact between an electrode and the user. For example, when the user is contacting an electrode, the measured test signal may be attenuated due to the user contact, and when the user is not touching the electrode, the test signal may not be attenuated (or may be attenuated less). In some cases, a threshold amplitude (e.g., voltage level) can be used to determine whether a sensing electrode is contacting the user. For example, when the measured test signal is less than a threshold amplitude, the device can determine that the sensing electrode is contacted (e.g., sufficient skin-to-electrode coupling exists for accurate physiological measurements). When the measured test signal is greater than or equal to the threshold amplitude, the device can determine that the sensing electrode is not contacted (or that there is insufficient skin-to-electrode coupling for accurate physiological measurements).

Additionally or alternatively, device may use other sensors to determine if the user is wearing the device and/or contacting the electrode. In some cases, the system may use temperature sensors, accelerometers, light sensors, moisture sensors, and so on. For example, the system may use a temperature sensor, which may include comparing temperature measurements to one or more thresholds/ranges. For example, if the measured temperature is within a defined range, the system may determine that the device and/or electrodes is in contact with the user, and if the measured temperature is out of the defined range the system may determine that the device and/or electrodes is not in contact with the user. In some cases, one or more temperature sensors may be positioned near an electrode, which may provide more accurate contact determinations. In other examples, light sensors can be positioned near an electrode (e.g., on a back sensor assembly near the electrodes) and be used to determine contact with the user. The light sensor may include light emitters that emit light toward the user and photodetectors, which receive a returned portion of the emitted light. The received light may be analyzed to determine contact with the user.

At operation 506, the process 500 can include determining a second contact metric for a sensing electrode based on contact of the electrode with the user. In some cases, the sensing metric can be an impedance and/change in amplitude (e.g., voltage level) for electrical signals that are transferred from the user's skin and to the electrode. Determining the second contact metric can include applying a test signal to the sensing electrode and measuring the resulting test signal using the contact detection circuit, as described herein. The system may analyze the measured test signal to determine the contact metric, which may be an input impedance to electrical signals based on contact with the user.

At operation 508, the process 500 can include measuring electrical signals of the user. For example, a physiological sensing circuit (e.g., physiological sensing circuit 304) may be used to measure cardiac electrical signals from a skin surface of a user. The cardiac electrical signals may be sensed by one or more electrodes, transferred to a buffer where the signal can be converted to a digital signal. In some cases, a gain adjustment can be applied to the digital signal and the signal can be transferred to a digital signal processor, which can be further analyzed and/or transferred to a host processor, which may cause the device to perform one or more functions based on the signal. For example, the digital signal processor and/or host processor may generate an ECG output signal, which shows changes in the polarization of a user's heart. The device may display the ECG signal on a display. Additionally or alternatively, the DSP and/or host processor (or remote processor) may determine physiological metrics such as beat-to-beat timing, a heart rate, heart rate variability, and/or identify conditions such as an arrhythmia, tachycardia, bradycardia, heart valve conditions and so on.

In some cases, the system may use the first contact metric and the second contact metric to adjust the measured electrical signals of the user. For example, the first and second contact metrics may each include an impedance and a ratio of the first input impedance and the second input impedance may be used to determine a gain adjustment that can be applied to the measured electrical signal from the user. The gain adjustment may increase amplitude of the measured electrical signal, which may improve the quality the quality of the measured signal prior to analyzing the signal by the DSP and/or host processor.

In some cases, the system may determine and apply a gain adjustment to each measured electrical signal from a user. For example, in response to initiating a sensing session, the system may determine a second contact metric, which is used to determine a gain adjustment for measured signals. Accordingly, different gain adjustments may be applied during different sensing sessions and be based on the electrode contact with the user. In other cases, the second contact metric may be periodically or continually checked and updated during a sensing session. Accordingly, the systems and devices herein may adapt as the contact (and impedance) between a user and an electrode changes.

In some cases, the system may only apply a gain adjustment when at least one impedance criterion is not satisfied (path 501) to account for high signal attenuation. If the at least one impedance criterion is satisfied, the signal attenuation may be low enough that the system can accurately determine physiological parameters without applying a gain adjustment (path 503) to the measured electrical signals.

At operation 510, the process 500 can include determining if at least one impedance criterion is satisfied. The at least one impedance criterion may be used to determine an attenuation and/or quality of the sensed cardiac signal. Determining whether the at least one impedance criterion is satisfied can include analyzing the second contact metric (e.g., determined at operation 506) and the first contact metric (e.g., determined at operation 502) to determine if the impedance for the measured electrical signal (e.g., measured at operation 508) is of sufficient quality to determine physiological parameters for a user.

In some cases, the at least one impedance criterion may be a defined threshold and the first and second contact metrics may be used to determine an impedance ratio. If the ratio is above the defined threshold, the system may determine that the second input impedance does not satisfy the at least one impedance criterion and may apply a gain adjustment to the measured electrical signals using path 501. If the ratio is below the defined threshold, the system may determine that the second input impedance satisfies the at least one impedance criterion and may processes the measured electrical signal without applying a gain adjustment using path 503.

At operation 512, in response to determining that the at least one impedance criterion is not satisfied, the process 500 may include determining a gain adjustment using the first and second contact metrics. For example, the first and second contact metrics may each include an input impedance for the sensing electrode. The system may analyze the first and second input impedances to determine a change in impedance between the baseline impedance (e.g., first input impedance determined during bassline/calibration operation at 502) and the second input impedance due to contact with a user. In some cases, the gain adjustment can be proportional to the difference between the first input impedance and second input impedance.

At operation 514, the process 500 can include determining a physiological parameter of the user using the determined gain adjustment. For example, the determined gain adjustment can be a digital gain adjustment that is applied to the measured electrical signal after it has been converted to a digital signal. The gain adjustment may be applied to the signal prior to the signal being transferred to the digital signal processor and/or a host processor. In other case, the gain adjustment may be applied to the analog signal prior to converting the signal to a digital signal (also referred to herein as an “analog gain adjustment”).

In some instances, the gain adjustment may include a combination of a digital gain adjustment and an analog gain adjustment. Because a digital gain adjustment will also amplify all upstream noise, including circuit noise introduced as the measured electrical signal is routed through the system, in some instances it may be desirable to limit the magnitude of the digital gain adjustment. Accordingly, the system may apply both an analog gain adjustment to the analog measured signal (e.g., by changing the gain of one or more amplifiers within the system) and a digital gain adjustment to the measured signal after it has been converted to the digital signal. In this way, the analog gain adjustment forms a first portion of the determined gain adjustment and the digital gain adjustment forms a second portion of the determined gain adjustment. The analog gain adjustment may reduce the relative impact of downstream circuit noise, but may increase the power consumption of the system. Accordingly, the relative amounts of analog gain and digital gain within the determined gain adjustment may be selected to balance noise reduction and power consumption. For example, in some variations the determined gain adjustment may include only a digital gain adjustment when the digital gain adjustment is below a threshold level, and may include both a digital gain adjustment and an analog gain adjustment when the digital gain adjustment is at or above the threshold level. In these instances, digital gain adjustments may be used for relatively small adjustments, and a combination of digital and analog gain adjustments may be used for relatively large adjustments.

At operation 516, in response to determining that the at least one impedance criterion is satisfied, the process 500 can include determining a physiological parameter of a user without applying a gain adjustment to the measured electrical signal as described herein.

Although FIG. 5 illustrates an example process for performing gain adjustment for a single electrode, it is understood that similar test signal circuitry can be integrated onto the signal path of one or more additional electrodes on the device. Additionally, although FIG. 4 illustrates two sensing electrodes 402, 404 and one reference electrode 405, in some examples, the system can have a additional or less sensing electrodes and/or a plurality of reference electrodes, and similar test signal circuitry can be integrated with some or all of these electrodes.

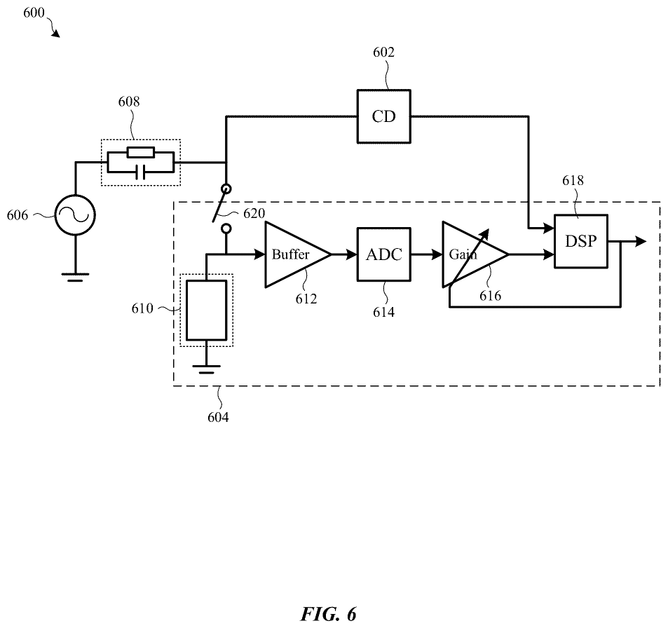

FIG. 6 shows an example gain calibration circuit 600 that can be used to perform physiological measurements, as described herein. The example circuit 600 may be an example of the circuits described herein (e.g., gain adjustment circuit 300 and circuit 400) and can include a contact detection (CD) circuit 602 and a physiological sensing circuit 604, as described herein. In some cases, the example circuit 600 may be implement on a single application specific integrated circuit (ASIC). In other cases, the example circuit 600 may be implemented across multiple ASICs and various components can be integrated on different ones of the multiple ASICs.

The measurements performed by a gain adjustment circuit (e.g., gain adjustment circuit 300) can include a total impedance that represents the combination of the electrode impedance (e.g., electrode impedance 308) and the circuit impedance (e.g., circuit impedance 310). When these impedances are measured together, the system may not be able to differentiate between changes to the electrode impedance (e.g., resulting from differences in how a user is contacting the electrode) and circuit impedance (e.g., resulting from wear, etc.).

The gain calibration circuit 600 can allow for an electrode impedance 608 to the measured independent from the physiological sensing circuit 604. Further, the gain calibration circuit 600 can measure the total input impedance to be measured (the combination of the electrode impedance 608 and the circuit impedance 610). Using the individual electrode impedance 608 and the total impedance, the system can determine the circuit impedance 610. Accordingly, the system can independently determine the electrode impedance 608 and the circuit impedance 610 in real time without requiring initial calibration information. For example, the circuit 600 impedance may be determined in response to initiating a measurement session (e.g., prior to measuring electrical signals of a user) and/or when the electrodes are not contacting a user.

The circuit 600 can include a switch 620 which can isolate the physiological sensing circuit 604 from the contact detection circuit 602. The total impedance of the gain calibration circuit 600 may include the electrode impedance 608 and the circuit impedance 610. The system/device can operate the switch 620 to selectively isolate the contact detection circuit 602 from the physiological sensing circuit 604 and couple the physiological sensing circuit 604 to the contact detection circuit 602.

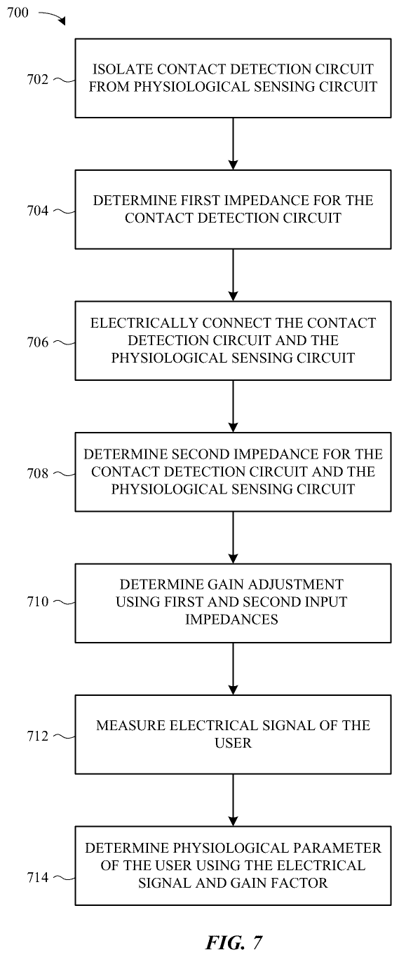

FIG. 7 shows a process an example process 700 for performing physiological measurements using gain calibration. The process 700 can be performed using the circuitry described herein (e.g., circuitry 600 described with reference to FIG. 6).

At operation 702, the process 700 can include isolating a contact detection circuit (e.g., contact detection circuit 602) from a physiological sensing circuit (e.g., physiological sensing circuit 604). In some cases, a switch (e.g., switch 620) can be operated to electrically disconnect the physiological sensing circuit from the contact detection circuit. Once isolated, the contact detection circuit can be electrically coupled to a sensing electrode and the physiological sensing circuit can be isolated from the contact detection circuit.

At operation 704, the process 700 can include determining a first impedance for the contact detection circuit. In some cases, the system can apply a test signal to a first node of the contact detection circuit and measure the resulting test signal at a second node of the contact detection circuit. The test signal may be changed (e.g., attenuated) due to impedance of the contact detection circuitry and/or the contact of the electrode with a user (or other object), as described herein. The system may determine the first impedance based on analyzing differences between the test signal applied to the contact detection circuit and the measured test signal. In some cases, the differences may include a change in amplitude, phase shift or other change to the test signal.

At operation 706, the process 700 can include electrically connecting the contact detection circuit and the physiological sensing circuit. The switch (e.g., switch 620) can be operated to electrically connect the physiological sensing circuit to the contact detection circuit. Once connected, the physiological sensing circuit can be electrically coupled to the contact detection circuit and the sensing electrode.

At operation 708, the process 700 can include determining a second impedance for the contact detection circuit and the physiological sensing circuit. The system can apply a test signal to a first node of the contact detection circuit and measure the resulting test signal at a second node of the contact detection circuit. The test signal may be changed (e.g., attenuated) due to impedance of the contact detection circuitry, impedance of the physiological detection circuitry and/or the contact of the electrode with a user (or other object), as described herein. The system may determine the second impedance based on analyzing differences between the test signal applied to the first node and the measured test signal. In some cases, the differences may include a change in amplitude, phase shift or other change to the test signal.

At operation 710, the process 700 may include determining a gain adjustment using the first and second input impedances. For example, the system may analyze the first and second input impedances to determine if signal attenuation meets an impedance threshold, which may be set by system requirements. Determining if the signal attenuation meets the impedance threshold may be determined by comparing the electrode impedance 608 and input impedance 610. When the signal attenuation does not meet the impedance threshold, gain adjustment can be applied at 616. In some cases, the gain adjustment can be proportional to the difference between the first input impedance and second input impedance.

At operation 712, the process 700 can include measuring an electrical signal of a user. For example, a physiological sensing circuit (e.g., physiological sensing circuit 604) may be used to measure cardiac electrical signals from a skin surface of a user. The cardiac electrical signals may be sensed by one or more electrodes, transferred to a buffer where the signal can be converted to a digital signal.

At operation 714, the process 700 can include determining a physiological parameter of the user using the electrical signal and the gain adjustment. In some cases, a gain adjustment can be applied to the digital signal and the signal can be transferred to a digital signal processor, which can be further analyzed and/or transferred to a host processor, which may cause the device to perform one or more functions based on the signal. For example, the digital signal processor and/or host processor may generate an ECG output signal, which shows changes in the polarization of a user's heart. The device may display the ECG signal on a display. Additionally or alternatively, the DSP and/or host processor (or remote processor) may determine physiological metrics such as beat-to-beat timing, a heart rate, heart rate variability, and/or identify conditions such as an arrhythmia, tachycardia, bradycardia, heart valve conditions and so on.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of determining spatial parameters, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, haptic outputs may be provided based on non-personal information data or a bare minimum amount of personal information, such as events or states at the device associated with a user, other non-personal information, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A method for electrode sensing at a wearable electronic device, the method comprising:
in response to determining that a user is wearing the wearable electronic device:
applying, using a contact detection circuit, a test signal to a sensing electrode; and
determining, using the contact detection circuit, an input impedance for the sensing electrode using the test signal;
determining a gain adjustment using the input impedance and a baseline impedance;
measuring, using a physiological sensing circuit, an electrical signal of the user using the sensing electrode; and
applying, using the physiological sensing circuit, the gain adjustment to the measured electrical signal; and
determining a physiological parameter of the user using the measured electrical signal having the gain adjustment applied.

2. The method of claim 1, further comprising:
in response to determining that the input impedance does not satisfy a criterion, determining the physiological parameter by applying the gain adjustment to the measured electrical signal; and
in response to determining that the input impedance satisfies the criterion, determining the physiological parameter without applying the gain adjustment to the measured electrical signal.

3. The method of claim 1, further comprising, prior to applying the test signal, performing a calibration process that uses the contact detection circuit to determine the baseline impedance for the sensing electrode.

4. The method of claim 3, wherein the physiological parameter is determined as part of a first sensing session and further comprising:
determining by the wearable electronic device to initiate a second sensing session, after completion of the first sensing session;
in response to determining to initiate the second sensing session:
applying a second test signal to the user;
measuring a second electrical signal of the user
determining a second gain adjustment; and
determining a second physiological parameter of the user by applying the second gain adjustment to the measured second electrical signal.

5. The method of claim 1, wherein the test signal is a time-varying electrical signal.

6. The method of claim 1, wherein measuring the electrical signal of the user comprises measuring a cardiac electrical signal of the user using the sensing electrode and at least a second electrode located on the wearable electronic device.

7. The method of claim 6, wherein the sensing electrode contacts a first limb of the user and the second electrode is configured to be contacted by a second limb of the user when the wearable electronic device is worn by the user.

8. The method of claim 1, wherein the sensing electrode is a dry electrode that contacts a skin surface of the user when the wearable electronic device is worn by the user.

9. The method of claim 1, further comprising:
in response to determining that the user is wearing the wearable electronic device:
applying a second test signal to a second sensing electrode; and
determining a second input impedance for the second sensing electrode using the second test signal;
determining a second gain adjustment using the second input impedance and the baseline impedance;
measuring a second electrical signal of the user using the second sensing electrode; and
determining the physiological parameter of the user by applying the second gain adjustment to the measured second electrical signal.

10. A method for electrode gain calibration of sensing electrode signals at a wearable electronic device, the method comprising:
isolating a contact detection circuit from a physiological sensing circuit;
applying, while the contact detection circuit is isolated from the physiological sensing circuit, a first test signal to a sensing electrode of the wearable electronic device;
determining a first input impedance for the sensing electrode using the first test signal;
connecting the contact detection circuit to the physiological sensing circuit;
applying, while the contact detection circuit is connected to the physiological sensing circuit, a second test signal to the sensing electrode; and
determining a second input impedance for the sensing electrode using the second test signal;
determining a gain adjustment using the first input impedance and the second input impedance; and
measuring an electrical signal of a user using the sensing electrode; and
determining a physiological parameter of the user by applying the gain adjustment to the measured electrical signal.

11. The method of claim 10, further comprising:
in response to determining that the gain adjustment does not satisfy a criterion, determining the physiological parameter by applying the gain adjustment to the measured electrical signal; and
in response to determining that the gain adjustment satisfies the criterion, determining the physiological parameter without applying the gain adjustment to the measured electrical signal.

12. The method of claim 10, wherein measuring the electrical signal of the user comprises measuring a cardiac electrical signal of the user using the sensing electrode.

13. The method of claim 10, further comprising converting the measured electrical signal to a digital signal, wherein applying the gain adjustment comprises applying a digital gain adjustment to the digital signal.

14. The method of claim 10, wherein the sensing electrode is a dry sensing electrode.

15. The method of claim 10, wherein the first test signal and the second test signal each include a time-varying electrical signal.

16. A system for electrode sensing at a wearable electronic device, the system comprising:
a housing configured to be coupled to a user;
a sensing electrode coupled to the housing and configured to contact the user; and
a processor configured to:
in response to determining that the user is wearing the wearable electronic device:
cause a test signal to be applied to the sensing electrode; and
determine an input impedance for the sensing electrode using the test signal;
determine a gain adjustment using the input impedance and a baseline impedance;
measure an electrical signal of the user using the sensing electrode; and
determine a physiological parameter of the user by applying the gain adjustment to the measured electrical signal.

17. The system of claim 16, further comprising a second sensing electrode coupled to the housing, wherein:
the sensing electrode is configured to contact a first limb of the user;
the second sensing electrode is configured to be contacted by a second limb of the user; and
the processor is configured to measure a second electrical signal of the user using the second sensing electrode; and
determine the physiological parameter using the second electrical signal.

18. The system of claim 17, wherein the processor is configured to:
determine a second gain adjustment for the second sensing electrode; and
determine the physiological parameter by applying the second gain adjustment to the measured second electrical signal.

19. The system of claim 16, wherein the processor is configured to:
in response to determining that the input impedance satisfies a criterion, determining the physiological parameter by applying the gain adjustment to the measured electrical signal; and
in response to determining that the input impedance does not satisfy the criterion, determining the physiological parameter without applying the gain adjustment to the measured electrical signal.

20. The system of claim 19, wherein:
the criterion comprises a defined impedance threshold; and
determining that the input impedance satisfies the defined impedance threshold comprises determining that a value of the input impedance is greater than the defined impedance threshold.
